(19) United States
(12) Patent Application Publication
(10) Pub. No.: US 2025/0137020 A1
(43) Pub. Date: May 1, 2025

(54) PREPARATION METHOD OF ECHINOCANDIN NUCLEUS

(71) Applicant: BIOCON LIMITED, Bangalore (IN)

(72) Inventors: Omkar Bhooshan PAI, Dombivili-East (IN); lshwar BAJAJ, Bangalore (IN); Aditya KULKARNI, Bengaluru (IN)

(21) Appl. No.: 18/689,550

(22) PCT Filed: Sep. 7, 2022
(86) PCT No.: PCT/IB2022/058392

§ 371 Date: Mar. 6, 2024

(30) Foreign Application Priority Data
Sep. 7, 2021 (IN) 202141040608

(51) Int. Cl. C12P 17/16; C12N 9/80; C12N 11/06

(52) U.S. Cl. CPC C12P 17/165; C12N 9/80; C12N 11/06

(57) ABSTRACT

The present invention relates to an immobilized deacylase in cross-linked cell aggregates, a preparation method and use thereof in deacylation of echinocandins. The immobilization of deacylase in cross-linked cell aggregates comprises the step of
Production of Deacylase by fermentation
Aggregation of cells
Cross-linking of the cells.

Present invention also relates to the use of the cross-linked cell aggregates of deacylase in deacylation of Echinocandin intermediates.

### RELATED APPLICATION

This application claims the benefit of priority of our Indian patent applications IN 202141040608 filed on Sep. 7, 2021, which is incorporated herein by reference.

### TECHNICAL FIELD

The present invention relates to an immobilized deacylase in Cross-linked cell aggregates, a preparation method and use thereof in deacylation of echinocandins. The immobilization of deacylase in Cross-linked cell aggregates comprises the step of.
Production of Deacylase by fermentation
Aggregation of cells
Cross-linking of the cells.

Present invention also relates to the use of the cross-linked cell aggregates of deacylase in deacylation of Echinocandin intermediates.

### BACKGROUND AND PRIOR ART OF THE DISCLOSURE

Echinocandins are a group of semisynthetic, cyclic lipopeptides with an N-linked acyl lipid side chain. The echinocandins act as non-competitive inhibitors of β-(1, 3)-D-glucan synthase, an essential component of the fungal cell wall that is not present in mammals. Inability of the organism to synthesize β-(1, 3)-D-glucan leads to osmotic instability and cell death. The drugs in the class are: Caspofungin, Micafungin and Anidulafungin.

Micafungin which is derived from FR901379 is a highly selective antifungal agent and an inhibitor of 1,3-β-glucan synthesis. However, Micafungin I intermediate (FR901379) is known to have hemolytic activity due to the long acyl side chain of palmitic acid. Therefore, FR901379 was deacylated by the acylase enzyme to give Micafungin II intermediate (FR179642) and then reacylated (by chemical synthesis route) at the free amino group to yield FR131535 which is converted to Micafungin by chemical modification active against most Candida and Aspergillus species.

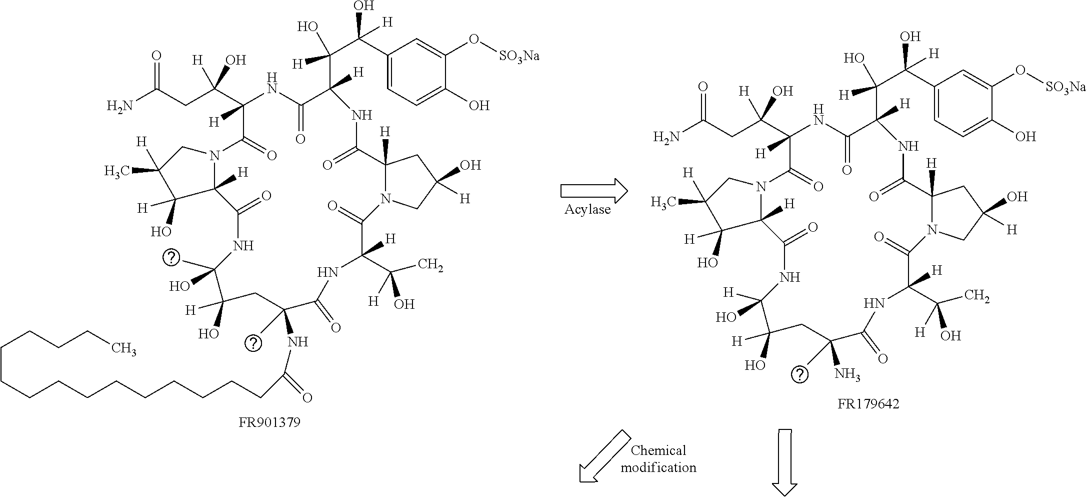

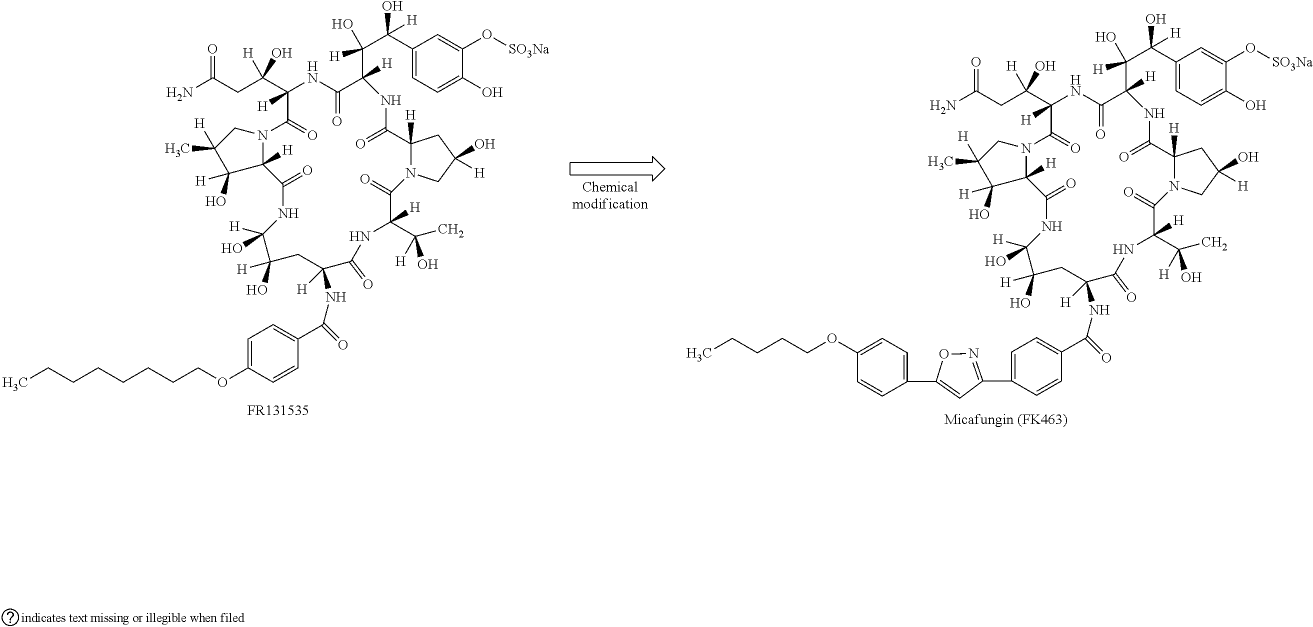

Echinocandin B (ECB) or Anidulafungin Intermediate-I is a lipopeptide antifungal agent produced by several species of Aspergillus. ECB can be modified by enzymatic deacylation to a cyclic hexapeptide without a linoleoyl side chain and by subsequent chemical reacylation to generate Anidulafungin.

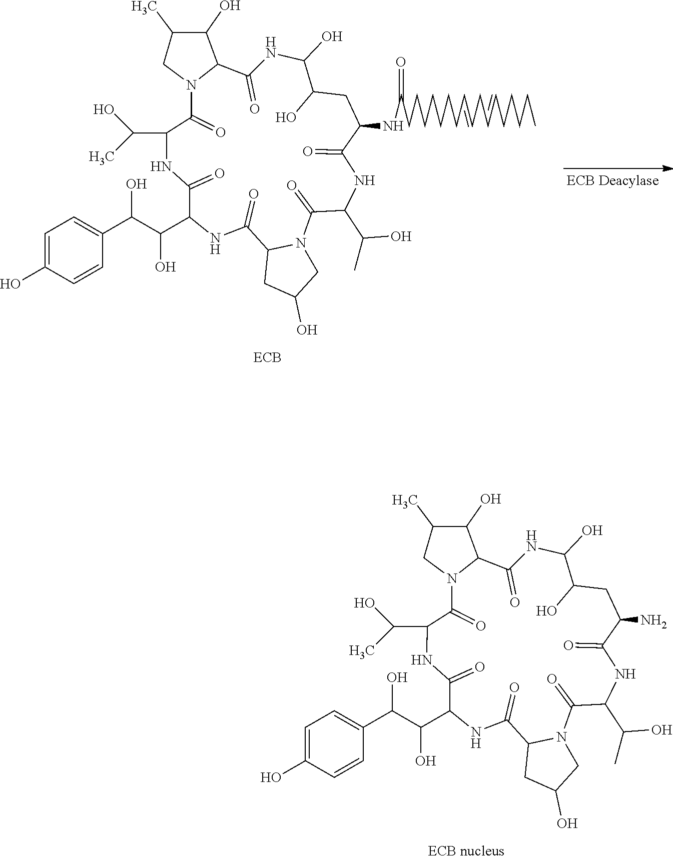 [SMILES: CCCCC/C=C/C/C=C/CCCCCCCC(=O)N[C@@H]1CC(O)C(O)NC(=O)C2C(O)C(C)CN2C(=O)C(C(C)O)NC(=O)C(C(O)C(O)c2ccc(O)cc2)NC(=O)C2CC(O)CN2C(=O)C(C(C)O)NC1=O ; CC(O)C1NC(=O)[C@H](N)CC(O)C(O)NC(=O)C2C(O)C(C)CN2C(=O)C(C(C)O)NC(=O)C(C(O)C(O)c2ccc(O)cc2)NC(=O)C2CC(O)CN2C1=O]

Actinoplanes utahensis is known to produce Deacylase or acyl transferase which removes the acyl unit from the amino terminus of Micafungin or anidulafungin I intermediate to yield the bio inactive cyclic peptide core, or “nucleus” (Micafungin or Anidulafungin Intermediate-II). Actinoplanes utahensis is a Gram-positive filamentous bacterium able to produce deacylases which hydrolyzes various aliphatic acyl-side chains of many antimicrobials, such as penicillins, lipopeptides, glycopeptides and capsaicin. The enzyme (produced by Actinoplanes utahensis) is a membrane-associated heterodimer composed of 63-kDa and 18-to-20-kDa subunits.

The existing art in the field for deacylation suffers from many issues are lower conversion rates, higher costs, and unsuitability for mass production etc.

U.S. Pat. No. 7,785,826 B2 discloses a process for ECB conversion of ECBN. The main flow of the process comprises the steps of: ECB fermentation, centrifuging to obtain mycelium, resuspending the mycelium in water, then adding the deacylase for conversion. This method utilizes the ECB deacylase for only one time. However, the method is complicated to operate, the process conversion time is 20-30 hours, the conversion rate is low, and the molar conversion rate is only 30%.

CN 102618606 discloses a method for bioconversion of echinocandins using actinomycetes whole cells or fermentation broth as a catalyst. The method has the advantages that the solubility of the substrate in the conversion system is improved, and the co-solvent is beta-cyclodextrin or a derivative thereof. The method has the advantages that the conversion speed and the conversion rate is improved, the defects are full cell transformation, the system has a large number of thalli, the contact efficiency of the enzyme and the substrate is very low, the subsequent separation and purification steps are complex, thus the cost is high, and the problem that the enzyme is prone to inactivation in an organic solvent system is used.

CN103387975 discloses a method for preparing an immobilized cycloaliphatic peptide acyltransferase, wherein the cycloaliphatic peptide acyltransferase is immobilized on a carrier; the cycloaliphatic peptide acyltransferase is derived from natural or artificial mutants, or variants, and transformed by introducing a foreign cyclic acyltransferase gene. The immobilized cycloaliphatic peptide acyltransferase is used to convert ECBN to anidulafungin. Through this method, although the conversion rate is high, the operation is complicated and the cost is high, and the chemical reaction of the immobilization process easily leads to partial inactivation of the enzyme.

In 2000, Cao et al. of Delft University of Technology in the Netherlands proposed a new type of immobilized enzyme technology based on cross-linked enzymes and cross-linked enzyme crystals, cross-linked enzyme aggregates (CLEAs). This changes the properties to bring the enzyme molecules close to form aggregates and precipitate them out of the solvent, and then cross-link the aggregates to form cross-linked enzyme aggregates. The cross-linked enzyme polymer is an immobilized enzyme with the enzyme itself as a carrier. The enzyme concentration per unit volume is high, stable, recyclable, high in catalytic activity, low in production cost, and has potential application prospects.

CN108676831A which uses the CLEA technique reports about 85% conversion rate for Echinocandin B into a Echinocandin B nucleus. The disadvantages of this invention are as below:
Need to isolate the enzyme from the microbial cells followed by preparing the Cross-Linked Enzyme Aggregates.
Purification of isolated enzyme from the fermentation broth needs to be performed.
Enzyme activity inhibition due to cross-linking at the enzyme active site is a limitation in case of CLEAs.
Process is not cost effective as process involves multiple steps for preparation and purification.
Reuse of CLEAs is limited in comparison to CLCA route.

### SUMMARY OF THE INVENTION

Although the deacylase-mediated biotransformation has exciting potential for the synthesis various antifungal agents, several challenges remain before it can be used industrially.

Particularly, its stability and reusability are relatively poor compared to many other industrial enzymes. These obstacles can be circumvented by the immobilization of enzymes. Cross-linked cell aggregates (CLCAs) as a carrier-free whole-cell immobilization method have a great potential for industrial application. Compared with carrier-bound immobilization technology, such as entrapment. adsorption and chemical binding, the activity of the carrier-free CLCAs is not diluted in the carrier. Moreover, the lack of necessity for cell lysis and purification step will reduce the cost of immobilization and simplify the manufacturing process.

The present invention discloses a method for the preparation of immobilized deacylase in Cross-linked cell aggregates (CLCAs) and use of the same for bioconversion. The advantages of the invention mainly include but not limited to:
The deacylase CLCAs can be repeatedly used, reducing production cost and facilitating industrial production.
The purity of the deacylated product of the present invention is significantly improved.
The process only uses cheap and easily available raw materials for immobilization and does not use any costly resins for immobilization. Hence, economical.
The CLCAs can be stored for a long period of time for re-use.
The bioconversion using CLCAs can be performed without the need for sterile/aseptic conditions unlike in case of fermented broth bioconversion.

### Advantages of the Present Invention Compared to Cross-Linked Enzyme Aggregates (CLEAs):

CLEA technology needs isolation of the enzyme from the microbial cells followed by preparing the Cross-Linked Enzyme Aggregates (CLEAs).
Directly Cross-linked cell aggregates (CLCAs) can be prepared and no multiple purification steps required for isolation of enzyme from the fermentation broth.
The Cross-linked cell aggregates (CLCAs) can be reused for multiple cycles of bioconversion.
Since, CLCAs involve cross-linking of cell surface proteins, enzyme activity inhibition due to cross-linking at the enzyme active site is overcome which is a limitation in case of CLEAs.
CLCAs enhance the stability of the enzyme under reaction conditions such as pH, temperature, shear stress due to mixing, etc. compared to CLEAs since the microbial cells act as support matrix for the enzyme.
Since preparation of CLEAs involve precipitation of the enzyme from its solution prior to cross-linking, partial loss of enzyme activity is a common limitation which can be overcome in case of CLCAs.

### Abbreviations

PCV: Packed Cell Volume
CLCA: Cross-Linked Cell Aggregates
CLEA: Cross-Linked Enzyme Aggregates
DMH: Dextrose Monohydrate
SF: Seed Flask
IF: Inoculum Flask
K₂HPO₄: Dipotassium hydrogen phosphate
PEI: Polyethyleneimine
GA: Glutaraldehyde
EOF—End of fermentation
EOR—End of Reaction
L—Litre
KL—Kilo Litre

One embodiment of the present invention discloses conversion of echinocandins into echinocandin parent nucleus. The method involves cross-linking deacylase cells and treatment with echinocandins to convert into desired echinocandin parent nucleus.

Wherein, the preparation method of the cross-linking deacylase cells involves:
a. Aggregation of deacylase cells
b. Cross-linking of aggregated cells
c. Isolation of cross-linked cells.

Another embodiment of the present invention discloses, conversion of Micafungin I intermediate (FR901379), into Micafungin II intermediate (FR179642). The method involves cross-linking deacylase cells and treatment with Micafungin I intermediate (FR901379) to convert into desired Micafungin II intermediate (FR179642).

Wherein, the preparation method of the cross-linking deacylase cells involves:
a. Aggregation of deacylase cells
b. Cross-linking of aggregated cells
c. Isolation of cross-linked cells.

Another embodiment of the present invention discloses, conversion of Echinocandin B, into Echinocandin B nucleus. The method involves cross-linking deacylase cells and treatment with Echinocandin B to convert into desired Echinocandin B nucleus.

Wherein, the preparation method of the cross-linking deacylase cells involves:
a. Aggregation of deacylase cells
b. Cross-linking of aggregated cells
c. Isolation of cross-linked cells.

Yet another embodiment of the present invention provides, a method for the conversion of echinocandins into echinocandin parent nucleus by treating cross-linked deacylase cells with echinocandins to yield desired echinocandin parent nucleus.

Wherein, the cross-linking of deacylase cells involves:
a. Aggregation of deacylase cells
b. Cross-linking of aggregated cells
c. Isolation of cross-linked cells.

Another embodiment of the present invention provides, a method for the conversion of Micafungin I intermediate (FR901379), into Micafungin II intermediate (FR179642) by treating cross-linked deacylase cells with echinocandins to yield desired echinocandin parent nucleus.

Wherein, the cross-linking of deacylase cells involves:
a. Aggregation of deacylase cells
b. Cross-linking of aggregated cells
c. Isolation of cross-linked cells.

Another embodiment of the present invention provides, a method for the conversion of Echinocandin B, into Echinocandin B nucleus by treating cross-linked deacylase cells with echinocandins to yield desired echinocandin parent nucleus.

Wherein, the cross-linking of deacylase cells involves:
a. Aggregation of deacylase cells
b. Cross-linking of aggregated cells
c. Isolation of cross-linked cells.

Yet another embodiment of the present invention provides, a method according to any of the preceding embodiments wherein, the aggregation step is performed using Polyethyleneimine.

Another embodiment of the present invention provides, a method according to any of the preceding embodiments wherein, the cross-linking step is performed using Glutaraldehyde.

### DETAILED DESCRIPTION OF THE INVENTION

The embodiments of the present invention are further described using specific examples herein after. The examples are provided for better understanding of certain embodiments of the invention and not, in any manner, to limit the scope thereof. Possible modifications and equivalents apparent to those skilled in the art using the teachings of the present description and the general art in the field of the invention shall also form the part of this specification and are intended to be included within the scope of it.

### EXAMPLES

### Example-1

### Step-1: Seed Fermentation

Seed Medium composition for SF, IF, seed fermenter stage

| S. No | Raw Materials | Concentration (g/L) |
|---|---|---|
| 1 | Sucrose A grade | 10 |
| 2 | Tapioca starch | 10 |
| 3 | Dextrose Mono hydrate | 10 |
| 4 | Cotton seed flour | 10 |
| 5 | Soya flour defatted & toasted | 5 |
| 6 | Soya peptone A grade | 5 |
| 7 | Calcium carbonate LR grade | 1 |
| 8 | Antifoam SAG 471 | 0.5 |

The seed fermentation medium comprises of the ingredients mentioned in the above table-1. All the ingredients were mixed and the pH was adjusted to 6.0±0.1 with 20% Sodium hydroxide (NaOH) or 20% Orthophosphoric acid (OPA) before sterilization. Inoculation was performed with ˜10% well grown inoculum in fermenter. The fermentation medium was transferred to production fermenter at age-approx. 96±24 h, PCV≥15 and when pH raised to around 7.3-7.5.

### Step-2: Production Fermentation

### Production Medium Composition for Production Stage

| S.No. | Raw materials | Concentration (g/L) |
|---|---|---|
| 1 | Sucrose | 20 |
| 2 | DMH | 10 |
| 3 | Yeast Extract A grade | 10 |
| 4 | Di-Potassium hydrogen phosphate LR grade | 1 |
| 5 | Potassium di hydrogen phosphate | 0.5 |
| 6 | Magnesium sulphate IH | 0.3 |
| 7 | Calcium carbonate LR grade | 0.5 |
| 8 | Antifoam SAG 471 | 0.5 |

The medium comprises of the ingredients mentioned in the above table-2. All the ingredients were mixed and the pH was adjusted to 6.0±0.2 with 10% Sodium hydroxide (NaOH) or 20% Orthophosphoric acid (OPA). Cooled the fermenter to a temperature to 25° C.±2° C. Inoculation was performed with ˜10% well grown inoculum. Sucrose was fed followed by DMH and yeast extract at 30 g/h starting from 6 h. Batch is expected to run for 96-120 h with PCV≥15%.

### Step-3: Cell Aggregation

After fermentation Polyethyleneimine (PEI) (0.2%) was added directly to the fermenter under constant mixing, keeping the pH of the broth at 6.8±0.2. Incubated the medium for 20-30 min at 25±2° C. at 100 rpm.

### Step-4: Cell Cross-Linking

After Cell aggregation step, Glutaraldehyde (GA) (0.4%) was added directly to the fermenter under constant mixing. Incubated the medium for 20-30 min at 25±2° C. at 100 rpm to form the Cross-Linked Cell Aggregates (CLCAs).

### Step-5: Cross-Linked Cell Aggregates (CLCAs) Filtration and Washing

Filtration of CLCAs was performed and CLCAs was washed twice with RO water to remove excess PEI & GA as well as fermentation broth. Filtered off and dried the CLCAs to remove excess of water.

### Protocol for CLCA Activity Testing

Prepare 50 mM solution of K₂HPO₄ with pH 5.5-6.0. Using the same buffer solution, prepare 10 g/L solution of Micafungin Intermediate-I. Add desired quantity CLCA solid to a conical flask. Transfer above prepared Micafungin Intermediate-I solution to the conical flask such that the final dilution of CLCA in the reaction mixture is around 10% w/w. Incubate the above mixture at 40° C. for about 60 min. Quench the reaction by adding o-phosphoric acid. Perform the required dilution with appropriate solvent and analyse for Micafungin Intermediate-II content by HPLC. The CLCA activity may range from 1.5 to 2.5 mg/g.

### Step-6a: Bioconversion of Micafungin I to II

20 g/L Micafungin I intermediate solution (on assay basis) in 0.05 M K₂HPO₄ buffer (pH 5.8±0.3) was prepared. Deacylase CLCAs was transferred to a reactor and mixed with Micafungin I intermediate solution in a reactor. RPM was maintained at 300±50 and temperature was kept at 25±3° C. throughout the process. After the completion of the reaction, reaction mixture was harvested and CLCAs were separated by filtration. The CLCAs were washed with 0.05 M K₂HPO₄ buffer (pH 5.8±0.3), filtered and dried.

### Bioconversion of Micafungin I to II

| | Conventional | | |
|---|---|---|---|
| | Fermentation | | Fermentation |
| Bioconversion Process | process | | with CLCA |
| Micafungin Intermediate-I | 4 | g/L | 25 g/L |
| Loading | | | |
| Micafungin Intermediate-II | ~3 | g/L | 20 g/L |

| | | |
|---|---|---|
| at EOF/EOR | | |
| Purity of Micafungin | 55-60% | 80-85% |
| Intermediate-II | | |
| Number of Days | 3-4 | 3-4 |
| Process requirement | Sterile operation | Non-sterile |
| | in fermenter | operation in reactor |
| Number of enzyme recycles | 0 | 5 |

### (Or)

### Step-6b: Bioconversion of Anidulafungin I to II

Deacylase CLCAs was transferred to a reactor and mixed with 0.05 M K₂HPO₄ buffer (pH 5.8±0.3). Anidulafungin I intermediate was added to make 12 g/L. RPM was maintained at 300±50 and temperature was kept at 25±3° C. throughout the process. After the completion of the reaction, reaction mixture was harvested and CLCAs were separated by filtration. The CLCAs were washed with 0.05 M K₂HPO₄ buffer (pH 5.8±0.3), filtered and dried.

### Bioconversion of Anidulafungin I to II

| | Conventional | | | |
|---|---|---|---|---|
| | Fermentation | | Fermentation | |
| Bioconversion Process | process | | with CLCA | |
| Anidulafungin | 3 | g/L | 10 | g/L |
| Intermediate-I | | | | |
| Loading | | | | |
| Anidulafungin | ~2 | g/L | 7 | g/L |

| | | |
|---|---|---|
| Intermediate-II | | |
| at EOF/EOR | | |
| Purity of Anidulafungin | 40-45% | 80-85% |
| Intermediate-II | | |
| Number of Days | 3-4 | 3-4 |
| Process requirement | Sterile operation | Non-sterile |
| | in fermenter | operation in reactor |
| | Methanol required | No requirement of |
| | for the reaction | methanol for the |
| | | reaction |
| Number of enzyme | 0 | 5 |
| recycles | | |

### Example-2

The above process for preparation of CLCA, bioconversion of Micafungin I to II and Anidulafungin I to II using CLCA was scaled up to kilo litre scale. Below is the scale up data for the same.

### Preparation of Cross-Linked Cell Aggregates (CLCA)

The process for preparation of CLCA was performed at 1 KL scale in fermenter and the process was validated. The CLCA preparation was done as per the protocol mentioned above in Example 1 (Step 1 to Step 5). The output details for the CLCA solid were as below.

| | CLCA Quantity | CLCA Activity |
|---|---|---|
| Scale | (kg) | (mg/g) |
| 1 KL | 91.9 | 1.33 |
| 1 KL | 93.4 | 1.48 |
| 1 KL | 80.7 | 1.22 |
| 1 KL | 87.2 | 1.90 |

Bioconversion of Micafungin I to II was performed at kilo litre scale and the process was validated. The results for the same are tabulated below.

| | Micafungin I | Micafungin II | Micafungin II |
|---|---|---|---|
| | Loaded | recovered | EOR Purity |
| Scale | (kg) | (kg) | (%) |
| 1 KL | 13.5 | 9.8 | 83.3 |
| 1 KL | 12.4 | 9.8 | 83.3 |
| 1 KL | 15.4 | 11.0 | 79.7 |

Bioconversion of Anidulafungin I to II was performed at higher scale. The results are tabulated below.

| | Anidulafungin I | Anidulafungin II at | Anidulafungin II |
|---|---|---|---|
| | Loaded | EOR | EOR Purity |
| Scale | (g) | (g) | (%) |
| 50 L | 70.0 | 47.0 | 83.8 |
| 50 L | 66.0 | 42.0 | 81.4 |
| 50 L | 60.0 | 40.0 | 88.5 |

1. A method for the conversion of echinocandins into echinocandin parent nucleus by treating cross-linked deacylase cells with echinocandins to yield desired echinocandin parent nucleus.
Wherein, the cross-linking of deacylase cells involves:
a. Aggregation of deacylase cells
b. Cross-linking of aggregated cells
c. Isolation of cross-linked cells.

2. A method for the conversion of Micafungin I intermediate (FR901379), into Micafungin II intermediate (FR179642) by treating cross-linked deacylase cells with echinocandins to yield desired echinocandin parent nucleus.
Wherein, the cross-linking of deacylase cells involves:
a. Aggregation of deacylase cells
b. Cross-linking of aggregated cells
c. Isolation of cross-linked cells.

3. A method for the conversion of Echinocandin B, into Echinocandin B nucleus by treating cross-linked deacylase cells with echinocandins to yield desired echinocandin parent nucleus.
Wherein, the cross-linking of deacylase cells involves:
a. Aggregation of deacylase cells
b. Cross-linking of aggregated cells
c. Isolation of cross-linked cells.

4. The method according to claim 1 wherein, the aggregation step is performed using Polyethyleneimine.

5. The method according to claim 1 wherein, the cross-linking step is performed using Glutaraldehyde.
